(19) United States
(12) Patent Application Publication
(10) Pub. No.: US 2014/0236294 A1
(43) Pub. Date: Aug. 21, 2014

(54) DEVICE AND METHOD FOR DETERMINING THE GRAFTING OF A RECIPIENT TISSUE SITE

(71) Applicant: Roger Khouri, Key Biscayne, FL (US)

(72) Inventor: Roger Khouri, Key Biscayne, FL (US)

(21) Appl. No.: 13/884,442

(22) PCT Filed: May 6, 2013
(86) PCT No.: PCT/US13/39670

§ 371 Date: May 9, 2013

Related U.S. Application Data
(60) Provisional application No. 61/643,030, filed on May 4, 2012.

(51) Int. Cl. A61B 5/00; A61F 2/12; A61B 5/03

(52) U.S. Cl. CPC A61B 5/4836; A61B 5/03; A61F 2/12; A61F 2002/48
USPC 623/8; 600/561

(57) ABSTRACT

A device comprising a kit and a method for using the kit are disclosed that warn a surgeon that the maximal grafting capacity of a recipient tissue has been reached. The increase in interstitial pressure is measured at the recipient site using any of several pressure sensor devices during the grafting process to alert the surgeon to stop grafting once a critical level has been reached, or a sudden increase in pressure is detected, indicative of a maximum graft size.

### BACKGROUND AND SUMMARY OF THE INVENTION

The grafting of live cells inside a recipient tissue site has numerous beneficial medical applications. These range from the grafting of islet cells to treat diabetes or the grafting of stem cells and muscle cells to treat heart failure, to the grafting of fat cells for the reconstruction of the breast after ablative surgery or its augmentation for cosmetic purposes. In all these situations, for the procedure to be successful, the cells in the injected graft have to survive. Crucial to the engraftment process is neovascularization. This is the process by which small capillary blood vessels from the recipient site sprout to reconnect with capillaries in the graft in order to restore its nutritive blood circulation.

The present inventor has extensive clinical experience with the grafting of fat tissue for the correction of soft tissue contour defect, and in particular the correction of post mastectomy defects and the augmentation of congenital small breasts. However, despite many promising results, the outcome in his hands and in the hands of expert surgeons all over the world has been found to be variable due to unpredictable fat resorption and necrosis. This has led the inventor to continue to search for any method or device that will increase graft survival and reduce the variability of result to thereby provide more reliably desirable results with these procedures.

The present inventor has previously submitted multiple patent applications on devices and methods to improve graft survival. These include his recently issued U.S. Pat. No. 8,066,691 B2, the disclosure of which is incorporated herein by reference. However, despite the best efforts of the inventor and other highly skilled surgeons working in this field, there remains a significant variability in results that previously could not be accounted for. The inventor thusly considered that a better understanding of the graft survival mechanisms was therefore necessary in order to further reduce this variability. In his continuing efforts to improve these procedures and obtain more reliable results, the inventor has come to understand that for a tissue graft to more reliably survive through efficient neovascular angiogenesis the surgeon should adhere to the following two fundamental principles.

First is the graft-to-recipient interface. See Matthew R. Kaufman et al.: Autologous Fat Transfer for Facial Recontouring: Is There Science behind the Art? Plast. Reconstr. Surg. 119: 2287, 2007. It is well known in the art that free transplanted fat, like any other tissue graft, needs to be in close contact with a well-vascularized recipient bed for survival. It has been found that cells further than 2 mm away from the recipient vasculature will likely die from lack of nutrients before the process of neovascularization can reach the central cell tissue. This close physical contact requirement between the recipient host vasculature and the graft means that collections larger than 2 mm should be avoided. The fat graft is thus preferably evenly and meticulously dispersed inside the recipient tissue as tiny individual micro-droplets squeezed inside surgically created micro-alveoli. The ingredient for success here is believed to be the precise, meticulous graft micro-injection to evenly and diffusely disperse the micro-droplets inside the recipient. See Kaufman, infra.

Second is the interstitial pressure. As fat is grafted, the recipient site volume increases commensurate with the graft volume which follows the additive formula:

Total Recipient Site Volume=Graft Volume+Recipient Volume.

Most recipient sites are distensible enough to accommodate a small volume increase. As more fat is progressively grafted, the recipient site gradually stretches to accommodate the volume increase. However, as graft volume increases further, the mechanical compliance of the recipient decreases. Beyond a certain point, the compliance of the tissue rapidly decreases and the interstitial pressure, a tightly guarded physiological value (Guyton 10), will suddenly increase. Continued grafting will lead to an interstitial pressure increase beyond physiologic range that will choke the microvasculature, precipitously drop capillary circulation, inhibit the delicate process of neovascularization and lead to graft failure. It is therefore at this point of detecting a sudden increase in pressure that a skilled surgeon will, using the present invention, be advised that continued grafting represents greatly increased risk of experiencing the undesirable issues of necrosis, etc. as explained above.

The inventor has found that even if the utmost care is taken in dispersing the microfat droplets, too much fat stuffed into too little space with limited compliance creates a choke effect and some, if not all, of the fat cells will not survive (FIG. 1). The inventor has thus discovered that the limit to how much graft a tissue can accommodate, even if extreme care is taken in the graft dispersion process, can be determined by monitoring the interstitial tissue pressure of the recipient as it accommodates the added graft volume. With small volume increases, the tissues are relatively compliant and the pressure increase is moderate; however as the volume increases to reach a tipping point, the compliance rapidly drops and the incremental pressure increase per % volume increase rises dramatically (FIG. 2).

It is not the absolute graft amount that matters, but rather how much is grafted into what size recipient. The inventor believes that the Percentage Volume Change is an accurate predictor to measure and control this process. Physiologists have previously measured the interstitial pressure increase per percentage volume change. Subcutaneous tissue is where edema accumulates, is where the body tends to sequester excess fluid, and is the most compliant tissue. Studies have shown that for the first 10% volume increase, intact non-scarred subcutaneous tissue interstitial pressure rises by about 2-4 mmHg. This is within the accepted physiologic range of interstitial pressure variability (Textbook of Medical Physiology). For the next 10% volume increase, the pressure rises by one mmHg per percentage increase. After the 20% volume increase, the pressure rapidly rises into a zone where necrosis becomes an issue. Thus, the present invention contemplates grafting until a predetermined pressure is reached, as those pressures are known in the art and as explained herein, or until a sudden increase in pressure is detected by the surgeon. Exemplary critical interstitial pressures may be found in the pathologic condition called compartment syndrome where the interstitial pressure rises above the pressure inside the lumen of the capillaries. This causes them to collapse and arrest capillary perfusion and blood flow to the tissues. It is generally accepted that capillary blood pressure is about 15 mmHg. Thus a rise in pressure that comes close to this value is considered dangerous, especially since post surgical trauma will tend to further increase the pressure.

In mega-volume grafting, it is the recipient site Percentage Volume Change that is believed by the inventor to be the most crucial factor. As 100 ml of fat grafted into a large 2000 ml buttock recipient represents only a 5% volume increase and is well within the range a buttock can accommodate without a significant pressure increase. If meticulously grafted using techniques as described above, that entire graft could theoretically survive at 100%. However, trying to graft the same 100 ml of fat in a scarred, radiated 200 ml mastectomy defect represents a 50% volume increase that will undoubtedly drive the interstitial pressure deep into the choke zone. Even with the most meticulous grafting and the very best graft material, this might result with not only total graft failure, but also necrosis of the recipient tissue and ulceration from the increased compartment pressure that totally choked capillary circulation.

The following formula is believed to explain this physiological phenomena:

Δp=Δv %/f

Where:

Δp is the interstitial tissue pressure change of the recipient area

Δv % is the percentage volume change of the recipient area

f is the tissue compliance.

At low Δv %, the tissues are distensible, the compliance f is high and as the Δv % increases the tissues become less and less distensible and the compliance drops. When the enveloping capsular sheath of the tissue or its internal interstitial fibers become completely taut, f (tissue compliance) drops to near zero and the Δp pressure increase rises dramatically with minimal Δv % changes.

Clinically, the fundamental problem is that the therapeutic window is very narrow. While a surgeon generally wishes to graft the most tissue possible, it is difficult to recognize when the threshold is reached where the last graft drop will cause the pressure to abruptly rise and thus endanger the entire graft. Furthermore, it is often where we need to graft the most that the scarred and limited sized recipient can accept the least. In practical terms, therefore, it is the inventor's belief that the crucial problem to be solved to ensure desirable results is knowing exactly when to stop grafting (FIG. 3).

While those of skill in the art agree that determining how much graft a recipient tissue can tolerate is a critical issue, to the best of the inventor's knowledge, to date no expert has used objective measurements of interstitial pressure rise as a monitoring tool. To stop grafting, most have relied on estimates of recipient site properties, turgor, tightness, blanching, or other subjective methods. It is thus the inventor's conception that measuring and monitoring the interstitial tissue pressure rise in the recipient during tissue grafting provides an objective way to determine when to stop and thus achieve reliable and consistently desirable results. Furthermore the inventor has developed a practical method for monitoring the pressure and applied it to 75 large volume fat grafting procedures. Comparing his results with a previous 600 sample of tissue grafting procedures where the device was not used he found that the incidence of graft failure was significantly less and that the procedure was much more reliable.

While the principal advantages and features of the invention have been briefly discussed above, a fuller understanding may be attained by referring to the drawings and detailed description which follows.

### BRIEF DESCRIPTION OF THE DRAWINGS

FIG. 1 illustrates progressive microdroplet fat injection increasing interstitial pressure;

FIG. 2 is a chart illustrating tissue compliance curves expected when grafting;

FIG. 3 is a graph illustrating expected graft survival versus graft quantity; and

FIG. 4 is a perspective view of the use of the invention during a fat grafting procedure.

### DETAILED DESCRIPTION OF THE PREFERRED EMBODIMENTS

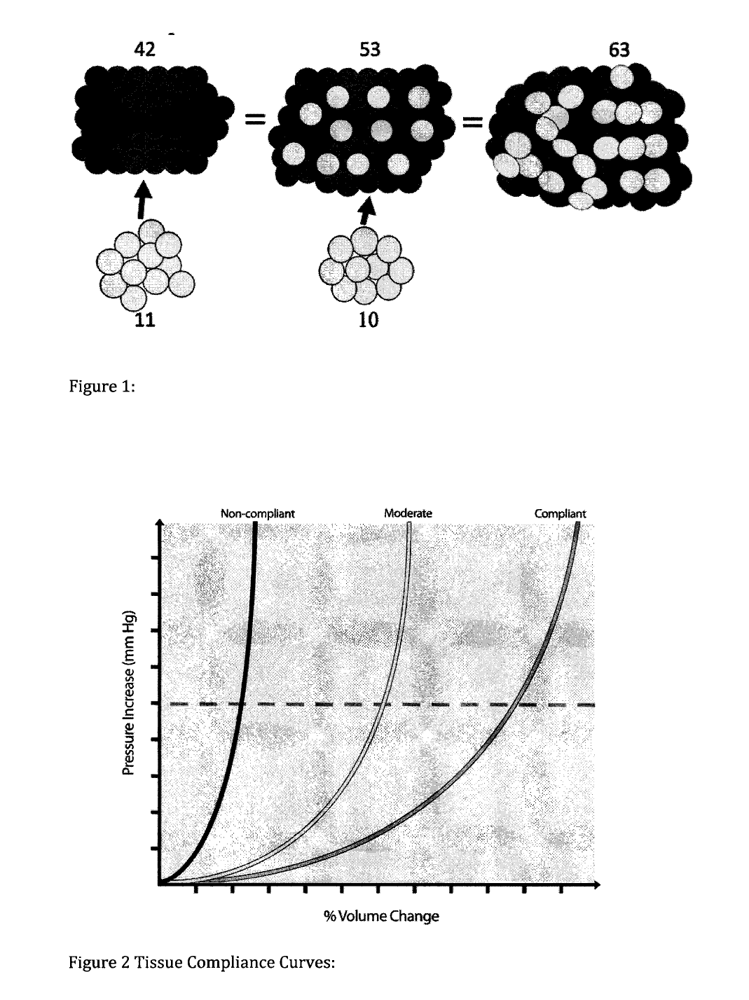

As shown in FIG. 1, 11 two-mm sized droplets may be evenly surgically grafted inside a 42 droplet sized recipient tissue. The recipient site is depicted as being distensible enough such that the interstitial tissue pressure does not increase significantly at the 53 droplets size and the meticulously inserted grafts may be expected to survive completely. However, squeezing another 10 droplets would likely result in 2 problems: 1—confluence; as droplets coalesce to become larger than 2 mm and end up with central necrosis; and 2—as the recipient tissue can no longer stretch and distend to accommodate the size increase, the interstitial pressure will rise to choke capillary circulation and prevent the neovascularization required for the graft to survive and cause additional necrosis. FIG. 1 thus illustrates that measurement of interstitial pressure can be used to guide a surgeon during grafting to achieve reliably desirable results.

As illustrated in FIG. 2, compliant tissue can accept more graft before a critical pressure limit is reached: Beyond a certain % volume change, compliance drops precipitously, the curve becomes nearly vertical and minimal additional grafting causes the pressure to dramatically increase. Tissues have different Volume to Pressure compliance curves, with subcutaneous being the most compliant, muscle intermediate, and scarred radiated tissue being the worst. By pre-expanding of the recipient, such as through use of the Brava® external expansion device 6, 7, the tissue compliance curve shifts to the right to thereby allow for greater amounts of graft. This device and method is the subject of previous patent filings by the inventor.

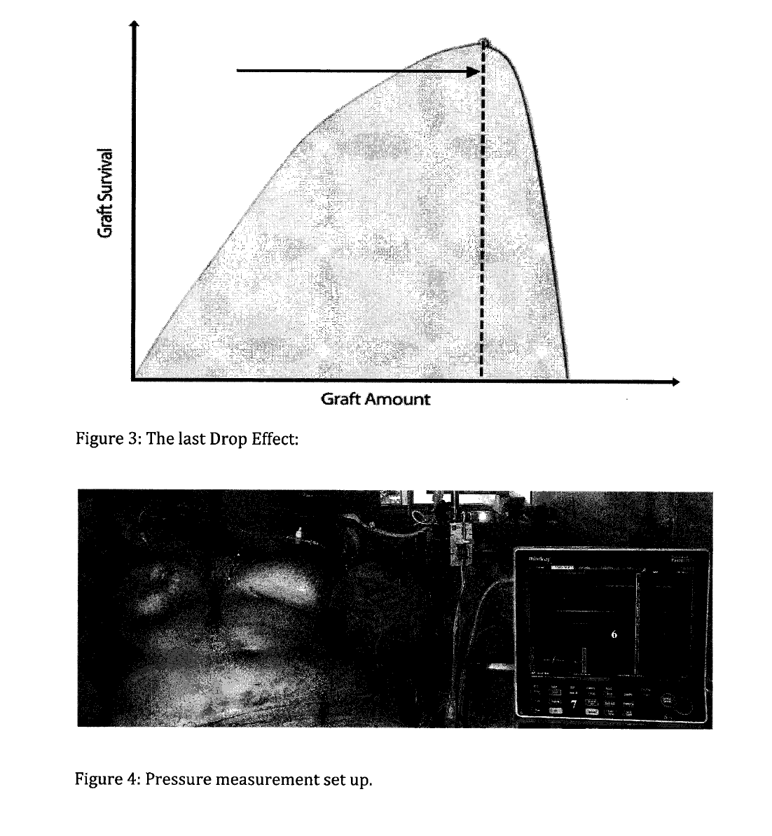

As illustrated in FIG. 3, there is a dramatic loss of fat graft survival when the last drop causes the interstitial pressure to exceed its maximally tolerable levels and graft survival to “fall off the cliff” (Vertical line). The larger and the more compliant the recipient the further to the right this vertical limit has been found to shift.

The use of the invention may be more fully understood by referring now to its use in a grafting procedure as depicted in FIG. 4. As depicted therein, a needle is inserted in the tissue to be grafted and the needle is connected through watertight sterile tubing to a pressure transducer. The transducer measures the equilibrium fluid pressure inside the tubing into which a minimal amount of physiologic fluid is slowly injected through a slow pumping mechanism. The transducer is connected to an electronic monitor that can display the measured pressure. To prevent hydrostatic pressure from affecting the reading, the pressure transducer is preferably level with the needle insertion site in the tissue. To that effect, a laser pointer with a leveling bubble is incorporated in the measuring device. As an alternative to the leveling device, some electronic monitors can be zeroed regardless of the relative position of the needle insertion to the pressure transducer. After flushing the line to eliminate air bubbles and ensure a continuous water column, the “Zero” pressure is set with the needle at the level of the tissue insertion point, with its lumen still exposed to the atmosphere. The needle is then inserted in the tissue and the equilibrium pressure read on the monitor display. Multiple samplings are often required as the pressure is not always homogenously distributed between the recipient site tissue compartments. Although there are multiple such devices on the market, an example of the pressure transducer that can be used is the Edwards® device commonly used in the intensive care units to monitor arterial and central venous pressures.

More particularly, as shown in FIG. 4, the measurement device is preferably “zeroed” with needle/catheter (1) just outside the skin at the level of the insertion. The needle is inserted in 4-6 random locations to sample the pressure in the various regions as the fibrous nature of the recipient does not necessarily allow for equilibration of the pressure in all areas. The inventor considers it important to sample and to make sure there is no area of excess pressure which would be undesirable. Once the catheter/needle (1) is inserted, the interstitial tissue fluid pressure is transmitted through the tubing (2) to the pressure transducer (3) and displayed on the electronic display monitor (6) connected to it via cable (5). Unless the monitor can electronically adjust to the “zero” (7) the pressure transducer is preferably brought to be substantially at the level of the tissue to be monitored with, for example, the help of a leveling device and a laser pointer (4).

Measuring compartmental pressure is known in the surgical art. A number of methods and devices have been advocated for that purpose. However, most if not all the clinically available devices are aimed at diagnosing a dreadful pathological condition known as compartment syndrome. This is a clinical situation where a feed-forward loop mechanism of tissue edema leads to increase interstitial tissue pressure within a confined tissue compartment leading to decrease tissue perfusion and ischemic injury which in turn leads to further edema, further pressure rise and further circulatory compromise and injury. The capillary lumen collapse and circulation come to a grinding halt when extrinsic interstitial pressure on the capillary wall is higher than the blood pressure inside the capillary.

Compartment syndrome typically happens in muscles with a well-defined, tight enveloping fibrous fascia sheath. As a result of strenuous exercise, vascular compromise, or reperfusion injury after ischemia, the muscles swell in the tight unyielding compartment and the interstitial tissue pressure rises to further compromise the circulation and worsen the situation until total necrosis ensues. This is often an emergency situation requiring an emergency operation called fasciotomy where the enveloping fascia is surgically incised to release the tightness and drop the interstitial pressures to the normal range while the tissue relieved from the non-compliant fascia freely swells. It is generally accepted that an emergency surgical fasciotomy should be performed when the interstitial tissue pressure comes close to 30 mmHg or comes within 30 mmHg of the diastolic arterial blood pressure.

The nascent neovasculature responsible for graft survival, however, is much more fragile and the inventor has recognized in his work that the critical pressure cut off point to stop grafting should be significantly lower. Also pointing to a much lower cut off point is the fact that grafting is in itself an edema causing traumatic process which will subsequently lead to an additional increase in pressure. On the other hand, depending upon the osmolarity of the graft and its water content, a certain degree of fluid shifts is likely to also alter the interstitial pressure.

It is recognized that depending upon the measurement technique used, the normal physiologic range of tissue interstitial pressure goes up to 4-6 mmHg. The inventor's clinical data points to the fact the pressure after grafting should not exceed 7-12 mmHg, depending upon the water content of the graft. This invention advocates monitoring of the interstitial tissue pressure during the grafting process and stopping when the pressure reaches the dangerous critical upper limit of 7-12, depending upon the water content of the graft (More dilute grafts can tolerate a higher pressure as excess fluid resorbtion is bound to bring the pressure down.)

A number of methods and devices have been advocated in the prior art to measure interstitial pressure. But in general terms these fall into the following categories or a combination thereof:
1—Those that measure the driving pressure required to inject very small amounts of additional fluid inside the tissue. They require the insertion of a hollow needle or cannula inside the tissue and have it connected by a closed fluid channel to a pressure transducer. The non-compressible fluid channel then transmits the pressure from the cannula aperture exposed to the interstitial space to the pressure transducer. Of these, the Stryker® device is the most popular. Unfortunately the commercially available version is limited by the fact it is designed for use in the clinic, not in the operating room. While the needle to be inserted in the tissue and the internal fluid channels are sterile, the monitor and the pressure transducers are not and therefore without modifications cannot be handled in the sterile surgical field of tissue grafting. As an alternative to the Stryker device, commercially available sterile cannulas can be connected via long tubings from the sterile surgical to commercially available sensitive pressure transducers.
2—Those that use an inserted wick to collect the interstitial fluid and directly measure its pressure. The wick measurement is considered more cumbersome and while it is commonly used in research settings where it can also measure colloid oncotic pressure, it is not practical for clinical use.
3—Those that use external bladders or other means of measuring pressure-volume changes such as plethysmography. While these methods require no needle insertion and are less invasive, they are generally less precise. As a research tool, implanted chambers with compliant bladder walls have also been used.

It is clear that there are many described methods for measuring interstitial tissue pressure. They vary in their practicality and their precision and also in the type of pressure they measure. Some measure the purely mechanical pressure, while others take into account the colloid osmotic pressure. A person expert in the art, utilizing the teaching of this disclosure and invention, would clearly be able to substitute a measuring technique for another and as long as the method is standardized, still use that measurement method to determine that the grafting capacity of the recipient has been reached.

While all of these can be modified to monitor the recipient interstitial tissue pressure rise during the grafting process, the inventor presently favors the first method as the compromise between precision, practicality and cumbersomeness. However, others may have different preferences depending on their experiences and familiarity with other devices.

Various changes may be made without departing from the invention, as would be apparent to those of skill in the art, including that the inserted needle can be sharp or blunt, have end holes or side holes and be of different bore sizes. As long as the measurement procedure is standardized these will not significantly affect the end point result. Similarly, variation and modifications of the measurement apparatus will not affect the final outcome that accurate measurement of the pressure is crucial to the success of the technique.

What is claimed is:

1. A kit for determining an optimal graft for a particular recipient tissue site, said kit including a device for measuring an interstitial tissue pressure at said tissue site and a monitor to display the reading of said measuring device.

2. The kit of claim 1 wherein said measuring device comprises a pressure transducer.

3. The kit of claim 2 wherein said measuring device comprises a hydrostatic pressure transducer for connection with a tube to a needle for insertion into the tissue site.

4. The kit of claim 3 further comprising a leveling device for use in aligning the tissue site with the pressure transducer.

5. The kit of claim 3 wherein said monitor includes an adjustment to zero out for differences in elevation between a needle insertion point and the pressure transducer.

6. The kit of claim 3 further comprising a needle for insertion at said tissue site and a length of tubing to connect the needle to the pressure transducer.

7. A method for determining an optimal graft for a particular recipient tissue site using the kit of claim 1 comprising using the measuring device to measure an interstitial pressure at said site as a graft is introduced thereinto, comparing said measured pressure with a threshold interstitial pressure, and ceasing grafting when the measured interstitial pressure reaches said threshold pressure.

8. The method of claim 7 wherein said kit further comprises a pressure transducer as said measuring device, the method further comprising inserting a needle at said tissue site and connecting said needle to said monitor.

9. The method of claim 8 wherein said kit further comprises a hydrostatic pressure transducer as said pressure transducer, the method further comprising adjusting the monitor for any difference in elevation between the needle insertion point and the hydrostatic pressure transducer.

10. The method of claim 9 wherein adjusting the monitor includes leveling the monitor with the needle insertion point.

11. The method of claim 9 wherein adjusting the monitor includes zeroing out the monitor after it is connected to the needle and situated for use.

12. A method for determining an optimal graft for a particular recipient tissue site using the kit of claim 1 comprising using said measuring device to measure an interstitial pressure at said site as a graft is introduced thereinto, and ceasing grafting when the measured interstitial pressure begins to rapidly increase.

13. The method of claim 12 wherein said kit further comprises a pressure transducer as said measuring device, the method further comprising inserting a needle at said tissue site and connecting said needle to said monitor.

14. The method of claim 13 wherein said kit further comprises a hydrostatic pressure transducer as said pressure transducer, the method further comprising adjusting the monitor for any difference in elevation between the needle insertion point and the hydrostatic pressure transducer.

15. The method of claim 14 wherein adjusting the monitor includes leveling the monitor with the needle insertion point.

16. The method of claim 14 wherein adjusting the monitor includes zeroing out the monitor after it is connected to the needle and situated for use.

17. A method for determining an optimal graft for a particular recipient tissue site comprising measuring an interstitial pressure at said site as a graft is introduced thereinto, comparing said measured pressure with a threshold interstitial pressure, and ceasing grafting when the measured interstitial pressure reaches said threshold pressure.

18. The method of claim 17 further comprising inserting a needle at said tissue site and connecting said needle to a monitor to display said measured pressure.

19. The method of claim 18 further comprising using a hydrostatic pressure monitor and adjusting the monitor for any difference in elevation between the needle insertion point and the hydrostatic pressure transducer.

20. The method of claim 19 wherein adjusting the monitor includes leveling the monitor with the needle insertion point.

21. The method of claim 19 wherein adjusting the monitor includes zeroing out the monitor after it is connected to the needle and situated for use.

22. A method for determining an optimal graft for a particular recipient tissue site comprising measuring an interstitial pressure at said site as a graft is introduced thereinto, and ceasing grafting when the measured interstitial pressure begins to rapidly increase.

23. The method of claim 22 further comprising inserting a needle at said tissue site and connecting said needle to a pressure monitor to display said measured pressure.

24. The method of claim 23 further comprising using a hydrostatic pressure transducer and adjusting the monitor for any difference in elevation between the needle insertion point and the hydrostatic pressure transducer.

25. The method of claim 24 wherein adjusting the monitor includes leveling the monitor with the needle insertion point.

26. The method of claim 24 wherein adjusting the monitor includes zeroing out the monitor after it is connected to the needle and situated for use.
